(19) United States
(12) Patent Application Publication
(10) Pub. No.: US 2009/0264724 A1
(43) Pub. Date: Oct. 22, 2009

(54) SERUM SAMPLING APPARATUS AND CATHETER

(75) Inventors: Stephen D. Walker, Boulder, CO (US); Peter E. Nelson, Boulder, CO (US); R. Dale Zellers, Boulder, CO (US); Charles W. Henry, Boulder, CO (US); John E. Repine, Boulder, CO (US)

Correspondence Address: KOESTNER BERTANI LLP, 2192 Martin St., Suite 150, Irvine, CA, 92612, US

(73) Assignee: Nostix LLC

(21) Appl. No.: 12/105,210

(22) Filed: Apr. 17, 2008

(51) Int. Cl. A61B 5/153; A61B 5/1473

(52) U.S. Cl. 600/345; 600/581; 600/583

(57) ABSTRACT

In a serum measurement device, an analyte concentration measurement apparatus facilitates sampling and analysis of analytes in body fluid and includes an implantable serum sampling catheter comprising a biocompatible tubing enclosing a vacuum release lumen and a serum lumen that are interconnected by a port. The serum lumen is separated from the sampling catheter exterior by a membrane barrier. The sampling catheter is configured for drawing a serum sample from a body fluid compartment by creation of suction in the serum lumen.

### BACKGROUND OF THE INVENTION

1. Field of the Invention

The invention relates to blood fluid sampling devices and associated methods.

2. Relevant Background

In 2001, Grete Van den Berghe, MD, published a seminal study (Van den Berghe G, et al. Intensive Insulin Therapy in Critically III Patients. NEJM, Vol. 345, No. 19, Nov. 8, 2001) that demonstrates the significant medical benefits derived by maintaining an Intensive Care Unit (ICU) patient's blood glucose levels between 80 and 110 mg/dl through highly managed insulin therapy. In the ICU, glucose levels commonly rise above 300 mg/dl as a consequence of stress, organ failure, infection, trauma, shock or other factors. Importantly, blood glucose levels increase dramatically even in patients without impaired glucose tolerance or diabetes (healthy, non-diabetics). Administering insulin to maintain blood glucose levels in the target range improves patient outcomes, perhaps due to the anti-inflammatory effects of insulin since seriously-ill individuals predominantly have elevated levels of inflammation. The Van den Berge study demonstrated very significant improvements in patient mortality, morbidity and length of hospitalization by aggressively using insulin to maintain low blood glucose levels and to decrease inflammation. Remarkably, intensive insulin therapy also has been found to reduce in-hospital mortality by 34%, acute renal failure by 41%, bacteremia by 46%, blood transfusions by 50%, and polyneuropathy by 44%.

Furthermore, patients receiving intensive insulin therapy are less likely to require prolonged mechanical ventilation and intensive care, an outcome also observed in patients with stroke, heart attack, and burn leading to the general concept of a “diabetes of injury” (Krinsley, J.: Effect of an intensive glucose management protocol on the mortality of critically ill adult patients, Mayo Clin. Proc. 79:992-1000, 2004, and Van den Berghe, G.: How does blood glucose control with insulin save lives in intensive care? J. Clin. Invest. 114:1187-1195, 2004). Dr. Van den Berghe's initial findings are corroborated by many other studies in settings ranging from surgical ICUs (Furnary, A P, Zurr K J, et al, Continuous intravenous insulin infusion reduces the incidence of deep sternal wound infection in diabetic patients after cardiac surgical procedures. Ann Thorac Surg 67:352-362, 1999) to general hospital wards (Newton, C A, Young, S, Financial implications of glycemic control, Endocrine Practice, Vol. 12, 7/8 2006, p. 43-48) to organ transplantations.

Based on the significant financial savings realized by hospitals when aggressively controlling blood glucose levels and the remarkable improvement realized in patient care from doing so, hospital use of aggressive insulin management protocol is not pursued simply due to cost.

Current techniques for measuring analytes such as blood glucose in seriously ill patients do not allow sufficiently frequent measurements and are expensive.

Conventional intensive blood glucose monitoring is expensive. For each new reading a glucose test strip, alcohol prep pad, cotton swab, lancet and gloves are used. While the single-use components may cost $1.50 in total on the open market, handling the components from a hospital's loading dock, through incoming inspection, into inventory, up to the ICU, onto a cart, to the patient's bedside and then disposed of after use, all the while complying with hospital tracking requirements, adds significantly to cost. An hourly glucose reading is then collected through one of two scenarios. The first involves dedicating one person per every 10 to 12 ICU beds to do nothing but collect blood glucose samples. The second uses the ICU nurse to take the reading. In the second scenario, the ICU nurse has one to two patients and collects an hourly blood glucose sample as part of standard care. Even for an experienced nurse, the test takes three to four minutes to prepare the site, make the blood stick, collect the blood sample, apply cotton, work the monitor, chart the value and dispose of the bloody material and wrapping materials. In either scenario, the fully burdened cost per glucose reading is $5 to $10, a new glucose value is generated only once every hour per patient and that value provides only a single data point of information from which to adjust insulin delivery rates. No method is known for real-time assessment of the glucose level's direction or rate of change, which is the critical information for aggressively and confidently managing insulin therapy.

While intensive insulin therapy programs cost more in labor and material to implement, the resulting savings in terms of shortened length of stay and fewer complications have been shown to result in a net savings of $40,000 per ICU bed per year (The ACE/ADA Task Force on Inpatient Diabetes, American College of Endocrinology and American Diabetes Association Consensus Statement on Inpatient Diabetes and Glycemic Control, Diabetes Care, Vol. 29, No. 8, 8/2006). Despite the savings and the improved outcomes, many medical and surgical ICU's cannot embrace the approach because intensive insulin therapy is difficult to accomplish in terms of staffing, training, implementing and managing.

### SUMMARY OF THE INVENTION

In accordance with embodiments of a serum measurement device, an analyte concentration measurement apparatus facilitates sampling and analysis of analytes in body fluid and includes an implantable serum sampling catheter comprising a biocompatible tubing enclosing a vacuum release lumen and a serum lumen that are interconnected by a port. The serum lumen is separated from a body fluid compartment by a membrane barrier. The sampling catheter is configured for drawing a serum sample from the body fluid compartment by creation of suction in the serum lumen.

### BRIEF DESCRIPTION OF THE DRAWINGS

The features of the described embodiments believed to be novel are specifically set forth in the appended claims. However, embodiments of the invention relating to both structure and method of operation, may best be understood by referring to the following description and accompanying drawings.

FIG. 1 is a schematic block and pictorial diagram showing an embodiment of an analyte concentration measurement apparatus that facilitates sampling and analysis of analytes in body fluid.

FIGS. 2A and 2B are pictorial view depicting a side view and a cross-sectional view of an embodiment of a double lumen sampling catheter.

FIGS. 3A and 3B are a schematic block and pictorial view and an exploded perspective view depicting an embodiment of an analyte sensor that can be implemented as part of an implantable serum sampling apparatus.

FIGS. 4A, 4B, and 4D are perspective pictorial views illustrating embodiments of sampling catheters including hollow fibers that can be used in an implantable serum sampling apparatus. FIG. 4C is a cross-sectional pictorial view showing a cross-sectional through the catheter depicted in FIG. 4A.

FIGS. 5A and 5B are schematic pictorial side and cross-sectional views showing an embodiment of an implantable serum sampling apparatus that facilitates sampling and analysis of analytes in body fluid.

### DESCRIPTION OF THE EMBODIMENT(S)

In accordance with embodiments of a serum measurement device, an analyte concentration measurement apparatus comprises an implantable serum sampling catheter, an analyte sensor configured for coupling to and receiving a sample of filtered serum from the sampling catheter, and a display and control device. The display and control device is coupled to the analyte sensor that controls acquisition of serum, measurement of analyte concentration in the serum, and display of measurement results.

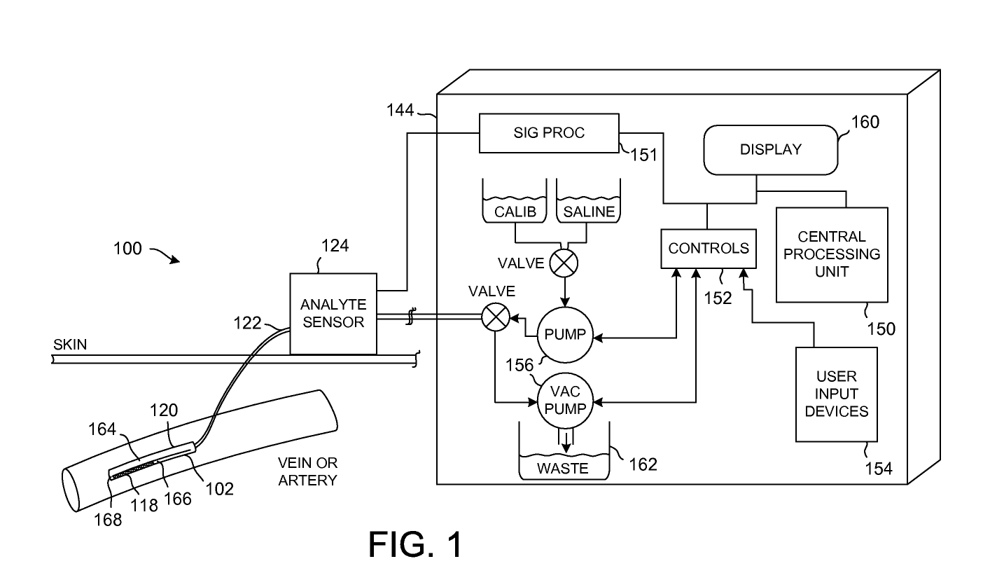

Referring to FIG. 1, a schematic block and pictorial diagram shows an embodiment of an analyte concentration measurement apparatus 100 that facilitates sampling and analysis of analytes in body fluid. The illustrative analyte concentration measurement apparatus 100 includes an implantable serum sampling catheter 102 comprising a biocompatible tubing 120 enclosing a vacuum release lumen 164 and a serum lumen 166 that are interconnected by a port 168. The serum lumen 166 is separated from a body fluid compartment by a membrane barrier 118. The sampling catheter 102 is configured for drawing a serum sample by creation of suction in the serum lumen 166.

The implantable serum sampling apparatus 100 can further comprise an analyte sensor 124 configured for coupling to and receiving a sample of filtered serum from the sampling catheter 102 and a display and control device 144. The display and control device 144 is coupled to the analyte sensor and performs various operations including controlling serum acquisition, measuring analyte concentration in the serum, and displaying measurement results.

In an example implantable serum sampling apparatus 100, the display and control device 144 comprises at least one pump 156, a control module 152, a processor 150, an analog signal processing module 151, and a display 160. The control module 152 receives user commands from one or more input devices 154 and sends control signals to the analyte sensor 124 and controls one or more pumps 156. The processor 150 is interfaced to the control module 152 and processes absorption measurement data for display. The signal processing module 151 receives measurement signals from the analyte sensor 124 and passes digitized absorption measurement data to the processor 150. The display 160 is coupled to the processor 150 and displays the processed absorption data.

The display and control device 144 can be implemented to operate in at least four modes including calibrate, infuse, sample, and measure modes. The calibrate mode operation comprises infusing a known analyte concentration to the analyte sensor 124 and calibrating the sensor 124 according to the known solution. In the infuse mode operation, saline is infused through the sampling catheter into the body compartment. In the sample mode operation, a serum sample is effused from the body compartment to the analyte sensor 124. The measure mode operation comprises measuring the analyte concentration in the serum sample.

In an illustrative embodiment, the display and control device 144 is configured to generate a display of the analyte concentration measurement in the serum sample within less than one minute of measurement initiation.

The display and control device 144 can further comprise a waste receptacle 162 coupled to the serum sampling catheter 102 that is configured for evacuating a sample of serum from the analyte sensor 124.

The illustrative sampling catheter 102 can be configured for usage in fluid drainage, fluid injection, instrument access, and/or separation of serum from whole blood.

The serum sampling system 100 can be implemented with three basic components that can be supplied independently or in combination. The components include a sampling catheter 102, an analyte sensor 124 which can be called an optical bench, and a display and control device 144, as shown in FIG. 1. The sampling catheter 102 is typically implanted in a vein or artery. The analyte sensor 124 can be attached to the patient's body near the sampling catheter connector 122. The display and control 144 can be positioned in a suitable position, for example at the bedside. The system 100 can have four modes of operation including calibrate, infuse, acquire, and measure. To calibrate the system 100, a solution of glucose and saline can be pumped from the display and control 144 into the analyte sensor 124 where a calibration procedure is performed. To flush the system 100, saline is pumped from the display and control 144, through the analyte sensor 124, through the sampling catheter 102 and into the blood stream. A flush is performed after every calibration to remove glucose from the sensor 124 and after every measurement to return serum to the patient and clear the sampling catheter 102 of obstructions that might prevent the next serum sample from being acquired. A glucose measurement is taken by activating the pump 156 to draw the previous serum sample into the waste receptacle 162 and a new serum sample into the analyte sensor 124. The glucose content of the new serum sample is measured with the analyte sensor 124 and transmitted to the display and control 144.

The sensor 124 can be an analyte sensor which is configured for connecting to the sampling catheter 102 and for mounting on a patient's body adjacent the sampling catheter 102. The illustrative sampling catheter 102 comprises a tubing 120 and a membrane barrier 118 which encases the tubing 120 and filters red blood cells from serum entering the tubing 120. The display and control device 144 draws a sample from filtered serum of less than 1 milliliter volume for measurement. Accordingly, the illustrative system 100 enables a small sample size since, in an illustrative implementation, less than 1 ml of serum can be removed from the patient for each measurement. The analyte sensor 124 typically uses on the order of 5 microliters (μl) of serum to make a measurement. The analyte sensor 124 is sufficiently small to be mounted on a patient's forearm, upper leg, shoulder or chest and the volume of serum within tubing 120 connecting the body to the analyte sensor 124 is less than approximately 0.45 ml.

The sampling catheter 118 has pores of diameter in a range of approximately 0.005-0.1 micrometers whereby blood components larger than the pore size are rejected by the membrane 118.

The display and control device 144 includes a controller 150 that displays a venous or arterial sample concentration measurement within less than one minute of measurement initiation. Accordingly, the illustrative system 100 enables acquisition with a short duration lag time.

The illustrative system 100 further enables frequent analyte measurements, such as glucose measurements. For example, glucose measurements can occur as frequently as about once every two minutes.

The implantable serum sampling apparatus 100 can further comprise a waste receptacle 162 coupled to the sampling catheter 102 that evacuates a sample of serum.

The system 100 enables complete avoidance of blood products waste since all fluids deposited in the waste receptacle are either calibration solution or saline, thus reducing or minimizing handling of biohazard materials.

The system 100 also can eliminate loss of red blood cells. Avoiding loss of red blood cells is particularly useful for patients with anemia, pulmonary disease, and the like.

In an illustrative embodiment, the display and control device 144 can comprise a control module 152 that receives user commands from one or more user input devices 154, sends control signals to the sensor 124 and sends pneumatic controls to one or more pumps 156, the signal processing module receives measurement signals from the sensor 124, and generates digitized absorption measurements. The display and control device 144 further comprises a processor 150 interfaced to the control module 152 that receives the digitized absorption measurements from the control module 152 and processes absorption data for display. A display 160 coupled to the processor 150 displays the processed absorption data.

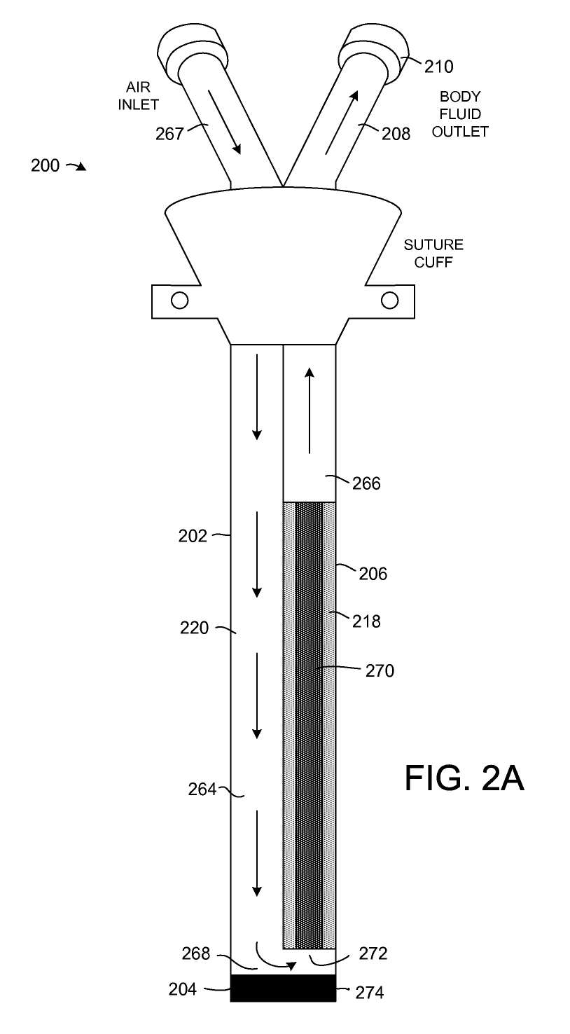

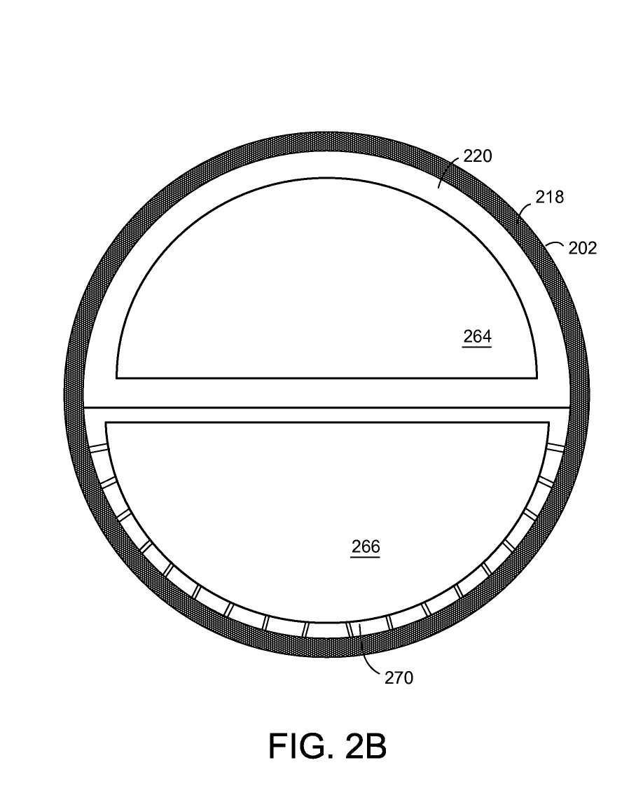

Referring to FIG. 2A, a pictorial view illustrates an embodiment of an implantable serum sampling apparatus 200 that facilitates measurement of analyte concentration. The illustrative serum sampling apparatus 200 comprises a sampling catheter 202 configured for insertion in an artery, vein or body fluid compartment. The sampling catheter 202 is formed from biocompatible tubing 220 extending longitudinally from a proximal end 210 to a distal tip 204 enclosing at least two lumens. The two or more lumens include a serum lumen 266 and a vacuum release lumen 264. The serum lumen 266 is circumferentially enclosed at least in part by a tubular membrane 218 and extends to a solid wall at the distal tip 204. The vacuum release lumen 264 is also circumferentially enclosed within the biocompatible tubing and extends to a solid wall at the distal tip 204. A port 268 in the biocompatible tubing 220 interconnects and enables air flow between the serum lumen 266 and the vacuum release lumen 264 proximal to the distal tip 204.

In some embodiments, the sampling catheter 202 can further comprise a porous support 270 configured to support the membrane 218 and prevent membrane collapse.

The illustrative implantable serum sampling apparatus 200 has two lumens in the double lumen sampling catheter 202, including the vacuum release lumen 264 and the serum lumen 266. The vacuum release lumen 264 has an air intake 267 coupled to a connecting passageway 268 to the serum lumen 266 near the tip section 204 of the double lumen sampling catheter 202. The serum lumen 206 includes a porous support 270 within the serum lumen 266 and is circumferentially encased by the membrane 218. The tip section 204 comprising a plastic cap 274 sealing the interior lumen 272 of both the serum and vacuum release lumen. The exit section 208 and a connector section at the proximal end 210 are formed of a biocompatible tubing 220.

In an illustrative implementation, the tubular membrane 218 has pores with diameter in a range of approximately 0.005-0.1 micrometers so that components in body fluid larger than the pore size are rejected.

In an illustrative embodiment, an implantable serum sampling apparatus 200 comprises a sampling catheter 202 that functions as a catheter for fluid drainage, fluid injection, and/or instrument access, and functions as a blood separator that separates serum from whole blood.

In accordance with another embodiment of an implantable serum sampling apparatus 200, a sampling catheter 202 comprises a biocompatible tubing 220 with an serum lumen 266 coupled to the biocompatible tubing 220 that is configured to separate the serum lumen from an artery, vein or other body compartment such brain ventricles or bladders. The sampling catheter 202 is configured to acquire an analyte by pulling serum from body fluid through the membrane barrier 218 with a pump connected to the serum lumen 266. Cross-flow filtering is accomplished by creating suction in the serum lumen 266. The serum flow rate in cross-flow filtering is significantly higher than dead-end filtering with no air inlet at the distal end, because no vacuum exists to overcome to pull the serum sample out. Dead-end filtering also results in a low serum flow rate because the pores in the membrane become obstructed. Accordingly, the sampling catheter 202 does not function in the manner of a conventional dialysis catheter which passes fluid through a membrane and acquires an analyte by osmosis. In contrast, the illustrative sampling catheter 202 passes air rather than a fluid that dilutes the sample. Accordingly, the illustrative implantable serum sampling apparatus 200 improves sampling accuracy over a dialysis system because the exact concentration of analytes in the blood is measured. The dilution which occurs in dialysis catheters is avoided, enabling a far smaller sample volume. For example, in an example configuration an accurate measurement can be made by sampling only a 3-5 microliter sample.

Referring to FIG. 2B, a cross-sectional view depicts an embodiment of the sampling catheter 202 wherein the vacuum release lumen 264 is contained interior to the biocompatible tubing 220. The serum lumen 266 is bounded in part by the biocompatible tubing 220 and in part by the membrane barrier 218. The porous support 270 supports the membrane barrier 218, preventing collapse.

Referring to FIG. 1 in combination with FIGS. 2A and 2B, the implantable serum sampling apparatus 200 can further comprise a pump 156 and a connector 122 configured for coupling the sampling catheter 202 inserted in the artery, vein or body fluid compartment to the pump 156. In an example implementation, the pump 156 can be a diaphragm pump or other suitable pump. The implantable serum sampling apparatus 200 can further comprise a controller 150 communicatively coupled to the pump 156 that operates the pump 156 to apply suction to the serum lumen 266. The controller 150 also operates the pump 156 to draw serum into the serum lumen 266 for acquisition of less than approximately 1 ml of serum.

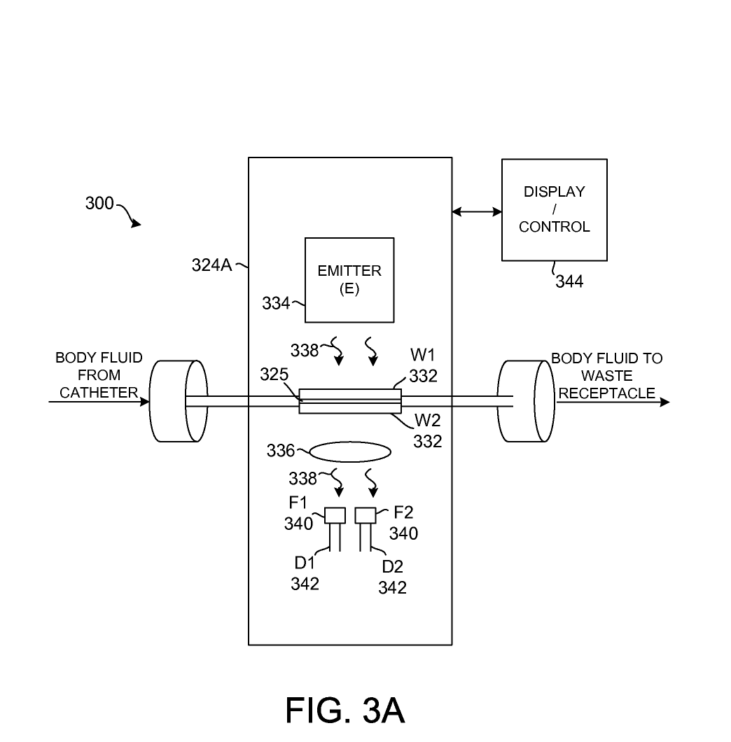

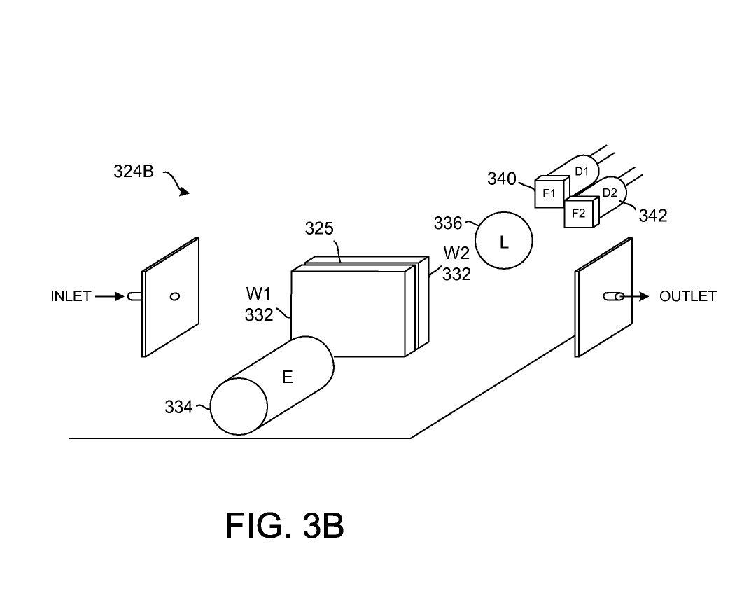

Referring to FIG. 3A in combination with FIGS. 1, 2A and 2B, the implantable serum sampling apparatus 100 can further comprise an analyte sensor 324A, operative as the analyte sensor 124, that is configured for connecting to the sampling catheter 102 and for mounting on a patient's body adjacent the sampling catheter 102. The illustrative analyte sensor 324A comprises one or more infrared windows 332, a broadband infrared emitter 334A that radiates through the infrared window or windows 332, a sample chamber 325, one or more narrowband filters 340 in the emission path of the emitter 334, and one or more detectors 342. The detector or detectors 342 receive infrared signals passing through the infrared windows 332, the sample chamber 325, the narrowband filter 340, and detectors 342 to transmit measurement signals to the display and control device 144.

Characteristics and operation of the sampling catheter 102 enable a compact configuration of the analyte sensor 324A that is suitable for mounting on a patient's limb, even for chronic monitoring. For example, the analyte sensor 324A can be configured for connecting to the sampling catheter 102 and accepting a sample of serum of less than 0.005 milliliter volume.

The analyte sensor 324A shown in FIG. 3A is sufficiently compact and lightweight to be attached to the patient. Measurement lag time is greatly reduced by placing the analyte sensor 324A at the sampling catheter connector. The analyte sensor 324A includes a broadband infrared emitter E 334 that radiates through infrared window W1 332, the serum sample, infrared window W2 332, lens L 336, narrowband filters F1, F2 340 and onto detectors D1 and D2 342. Absorption measurements are transmitted to the display and control 344. The sampling catheter connects to the analyte sensor input port. The previous sample of serum is evacuated through the output port to the waste receptacle.

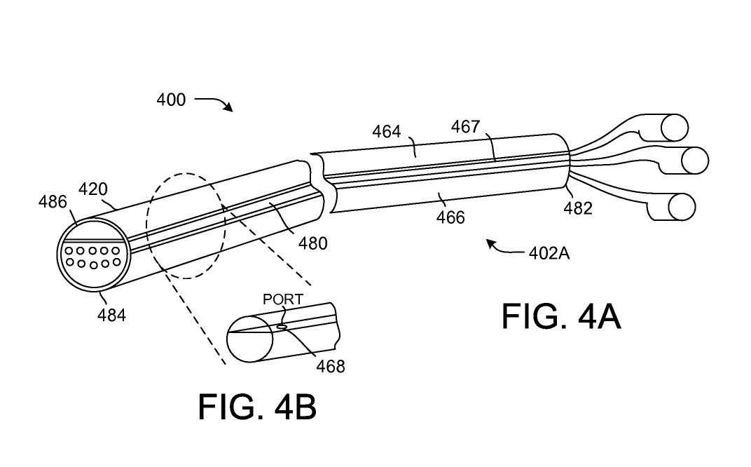

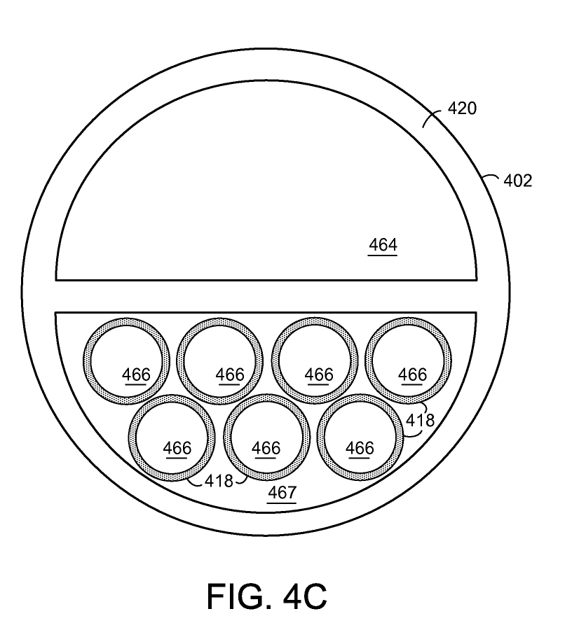

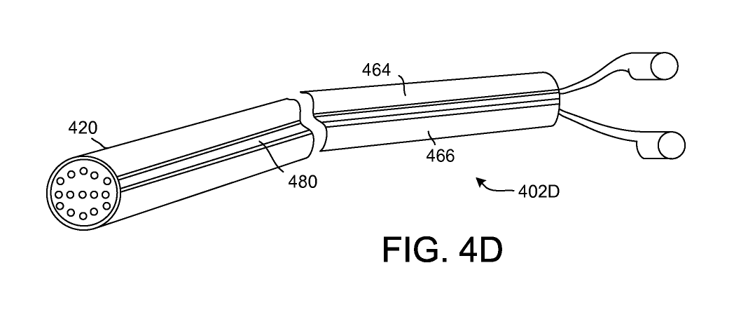

Referring to FIGS. 4A and 4B, perspective pictorial views illustrate respective embodiments of sampling catheters 402 (402A and 402C) that can be used in an implantable serum sampling apparatus. The illustrative implantable serum sampling apparatus 400 comprises a sampling catheter 402 that is configured for insertion in an artery, vein or body fluid compartment. The sampling catheter 402 comprises a biocompatible tubing 420 extending longitudinally from a proximal end 482 to a distal tip 484 enclosing at least three lumens. For example, the lumens can include a serum lumen 466, a vacuum release lumen 464, and a body fluid lumen 467. The serum lumen 466 is circumferentially enclosed within the biocompatible tubing 420 and extends to a solid wall 486 at the distal tip 484. The vacuum release lumen 464 is also circumferentially enclosed within the biocompatible tubing 420 and extends to a solid wall 486 at the distal tip 484. A port 468 in the biocompatible tubing 420 interconnects and enables air flow between the serum lumen 466 and the vacuum release lumen 464 proximal to the distal tip 484. The body fluid lumen 467 comprises a plurality of hollow fibers 480 extending longitudinally within the serum lumen 466 that are configured to receive body fluid from the artery, vein or body fluid compartment.

In an illustrative configuration, the hollow fibers 480 circumferentially contain pores with diameter in a range of approximately 0.005-0.1 micrometers so that body fluid components larger than the pore size are rejected.

Referring to FIG. 1 in combination with FIGS. 4A, 4B, 4C, and 4D, the implantable serum sampling apparatus 400 can further comprise a pump 156 with a volume displacement at least twice total interior volume of the body fluid lumen. A connector 122 couples the sampling catheter 402 which is inserted in the artery, vein or body fluid compartment to the pump 156. A controller 150 communicatively coupled to the pump 156 operates the pump 156 at a predetermined flow rate to draw a body fluid volume into the body fluid lumen 467, for example operating the pump 156 at a predetermine flow rate to evacuate a body fluid volume from the body fluid lumen 467.

The controller 150 can operate the system 100 to apply suction to the serum lumen 466 so that serum is drawn into the serum lumen 166 through pores in the hollow fibers 480 from the body fluid lumen 467, and release suction through the vacuum release lumen 464.

The perspective pictorial views shown in FIGS. 4A and 4B illustrate respective embodiments of sampling catheters 402A that can be used in an implantable serum sampling apparatus. The sampling catheter 402 comprises a biocompatible tubing 420 extending longitudinally that circumferentially encloses at least three lumens including a vacuum release lumen 464, a serum lumen 466, and a body fluid lumen 467. Multiple hollow fibers 480 extend longitudinally interior to the serum lumen 467 which is configured to separate serum from body fluid. The vacuum release lumen 464 and the serum lumen 466 are connected by a port 468. Suction is applied to the serum lumen 466 that draws serum through the pores of a plurality of hollow fibers wherein serum is separated from body fluid.

FIG. 4A depicts three connectors that enable coupling to the three lumens. In an illustrative embodiment the third lumen 467 contains the multiple hollow fibers 480 in a bundle that are coupled to a connector and extend through the length of the catheter 402A and extending out the end of the catheter 402A at the tip. Lumens 464 and 466 are sealed at the catheter tip. A port 468 shown by an insert diagram in FIG. 4B, typically near the catheter tip, extends through tubing between lumens 464 and 466 enabling flow. Connection to the hollow fibers 480 enables fluid infusion and return flow of a body fluid sample. A vacuum can be applied to the lumen 466 to draw the serum from the body fluid lumen through the hollow fibers 480. The hollow fibers have a pore size of 0.005-0.1 micrometers. This prevents any body fluid components with a larger diameter than the pore size from entering the serum sample. One of the lumens 464 can be opened, for example to air, and the sample is drawn out through the lumen 466, which is facilitated by the port 468 between the lumens 464 and 466 at the catheter tip. The configuration simplifies removal of the serum sample from the catheter 402A.

FIG. 4C is a pictorial view illustrating a cross-sectional through the catheter 402 depicted in FIG. 4A and shows a biocompatible tubing 420 and interior lumens including vacuum release lumen 464 and body fluid lumen 467. The body fluid lumen 467 contains multiple hollow fiber membranes 418 that filter serum from the body fluid. Internal to the hollow fiber membranes 418 are serum lumens 466.

Referring to FIG. 3B in combination with FIGS. 1, 4A and 4B, the implantable serum sampling apparatus 100 can further comprise an analyte sensor 324B that is sized and configured for connection to the sampling catheter 402 and mounting on a patient's body in a location that can be adjacent or near to the sampling catheter 402. The analyte sensor 324B includes a broadband infrared emitter 334 and at least one detector 342, forming an emission pathway 338 from the emitter 334 to detector or detectors 342. Arranged within the emission pathway 338 are at least one infrared window 332, at least one narrowband filter 340, and a sample chamber 325 arranged between the window or windows 332, and the narrowband filter or filters 342. The detector(s) 342 receive infrared signals passing through the infrared window(s) 332, the sample chamber 325, and the narrowband filter(s) 342 and transmit measurement signals to a display and control device.

The analyte sensor 324B is configured for connecting to the sampling catheter 402 and accepting a sample of serum of less than 0.005 milliliter volume.

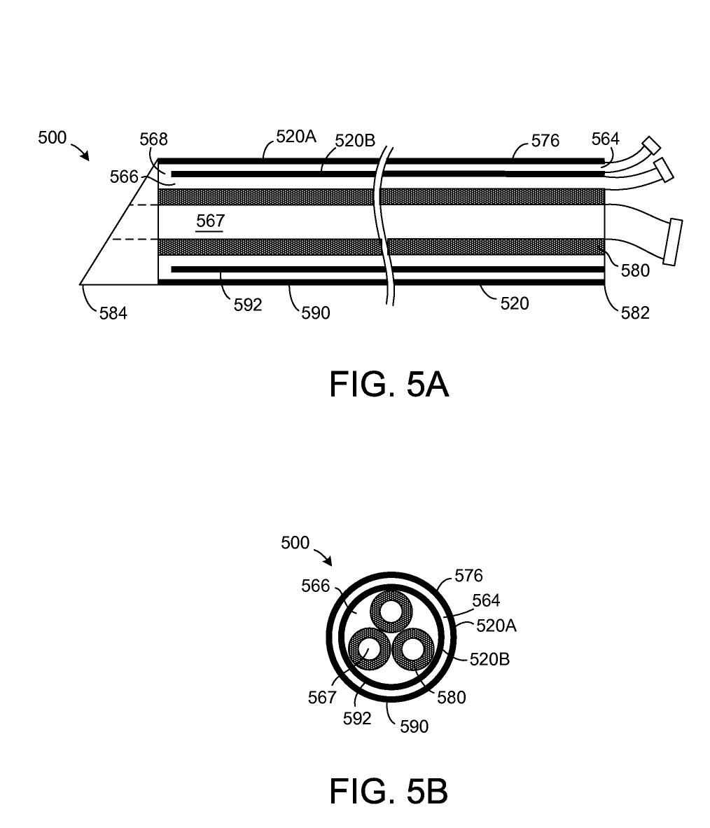

Referring to FIGS. 5A and 5B, schematic pictorial side and cross-sectional views show an embodiment of an implantable serum sampling apparatus 500 that facilitates sampling and analysis of analytes in body fluid. The implantable serum sampling apparatus 500 comprises an infusion needle 576 comprising at least one hypodermic tubing 520A, 520B enclosing a plurality of internal lumens. The internal lumens include a vacuum release lumen 564 and a serum lumen 566 that are interconnected by a port 568, and at least one body fluid lumen 567. The serum lumen 566 is separated from the body fluid lumens 567 by one or more hollow fibers 580 configured to receive body fluid from an artery, vein, or body fluid compartment. The infusion needle 576 is configured to draw a serum sample from the artery, vein, or body fluid compartment by creation of a pressure differential between the vacuum release lumen 564 and the serum lumen 566.

In the illustrative implantable serum sampling apparatus 500, the hypodermic tubing 520 extending longitudinally from a proximal end 582 to a distal tip 584 and includes an outer tubing 590 and an inner tubing 592 which is positioned internal to the outer tubing 590. The distal tip 584 of the hypodermic tubing 520 is configured for implanting in the artery, vein, or body fluid compartment. In the illustrative example, the vacuum release lumen 564 is enclosed between the outer tubing 590 and the inner tubing 592 with both distal tips sealed from the body fluid. The one or more hollow fibers 580 are positioned internal to the inner tubing 592. The serum lumen 566 is enclosed between the inner tubing 592 and the hollow fiber(s) 580. The medial serum lumen 566 and outer vacuum release lumen 564 are connected by the port 568 at the distal tip 584, enabling air flow between the serum lumen 566 and the vacuum release lumen 564. The one or more body fluid lumens 567 are contained within the one or more hollow fibers 580 and are configured to enable flow of body fluid from the artery, vein, or body fluid compartment. The one or more hollow fibers 580 function as a cross-flow separator that separates and filters body fluid from serum.

In an example embodiment, the hollow fibers 580 contain pores with diameter in a range of approximately 0.005-0.1 micrometers so that body fluid components larger than the pore size are rejected.

Referring to FIGS. 5A and 5B in combination with FIG. 1, the implantable serum sampling apparatus 500 can further comprise a pump 156 with a volume displacement at least twice the total interior volume of the one or more body fluid lumens 567. In an illustrative embodiment, the pump 156 can be a diaphragm pump. The implantable serum sampling apparatus 500 can further comprise a connector 122 configured for coupling the infusion needle 576 inserted in the artery, vein or body fluid compartment to the pump 156 and a controller 150. The controller 150 is communicatively coupled to the pump 156 and operates the pump 156 at a predetermined flow rate to draw a body fluid volume into the one or more body fluid lumens 567 and operates the pump 156 at a predetermined flow rate to evacuate a body fluid volume from the body fluid lumen(s) 567. The controller 150 can also be configured to apply a pressure differential between the serum lumen 566 and the vacuum release lumen 564 so that serum is drawn into the serum lumen through the hollow fibers 580 from the body fluid lumen(s) 567.

Referring again to FIG. 1, one or more embodiments or aspects of a method for sampling and analyzing body fluid comprise providing a sampling tubing 120, for example a biocompatible tubing, a catheter, a hypodermic tubing, a needle such as an infusion needle, and the like, enclosing a vacuum release lumen 164 and a serum lumen 166 that are interconnected by a port 168. The serum lumen 164 is separated from the sampling tubing exterior by a membrane barrier 118. The method further comprises drawing a serum sample from a body fluid compartment by creating a pressure differential between the vacuum release lumen 164 and the serum lumen 166.

In another aspect, a method can further comprise implanting the sampling catheter 102 into a patient's vein, artery, or body fluid compartment, and connecting the sampling catheter 102 to a sensor 124. During operation, the method can further comprise calibrating the sensor 124, acquiring an analyte, measuring the analyte in a serum sample, and flushing the sensor 124. The sensor 124 can be calibrated by infusing a known solution to the sensor 124 and calibrating the sensor 124 according to the known solution. The analyte can be acquired by effusing a body fluid sample from the vein or artery to the sensor 124. The sensor 124 can be flushed after each calibration and each measurement by infusing saline through the sensor 124 and the sampling catheter 102 into the vein or artery.

According to a further aspect and according to FIG. 1 in combination with either of FIG. 3A or 3B, a method can further comprise connecting the sampling catheter 102 to an analyte sensor 324A, 324B. The sampling catheter 102 can comprise a tubing 120 and a red blood cell-filtering barrier 118 encasing the tubing 120, for example in the form of hollow fibers that filter red blood cells from body fluid entering the tubing 120. The red blood cell-filtering barrier 118 can have pores of diameter in a range of approximately 0.005-2.5 micrometers whereby blood components larger than the pore size are rejected by the barrier 118. The method further comprises mounting the analyte sensor 124 on a patient's body adjacent the sampling catheter 102, and drawing a sample from filtered body fluid of less than 0.5 milliliter volume for measurement. The combination of the innovative sampling catheter 102 and analyte sensor 124 enable sampling of a small amount of serum drawn from the body compartment which is sufficient for highly accurate analyte measurements while enabling usage of compact, body-mountable measurement hardware that is convenient for both the patient and health care provider, even for chronic application.

While the invention has been described with reference to various embodiments, it will be understood that these embodiments are illustrative and that the scope of the invention is not limited to them. Many variations, modifications, additions and improvements of the embodiments described are possible. Those skilled in the art will readily implement the steps necessary to provide the structures and methods disclosed herein, and will understand that the process parameters, materials, and dimensions are given by way of example only and can be varied to achieve the desired structure as well as modifications which are within the scope of the invention. Variations and modifications of the embodiments disclosed herein may be made based on the description set forth herein, without departing from the scope and spirit of the invention as set forth in the following claims.

In the claims, unless otherwise indicated the article “a” is to refer to “one or more than one”.

What is claimed is:

1. A serum sampling apparatus comprising:
a sampling catheter configured for insertion in an artery, vein or body fluid compartment comprising:
a biocompatible tubing extending longitudinally from a proximal end to a distal tip enclosing at least three lumens, the at least three lumens comprising:
a serum lumen circumferentially enclosed within the biocompatible tubing and extending to a solid wall at the distal tip;
a vacuum release lumen circumferentially enclosed within the biocompatible tubing and extending to a solid wall at the distal tip;
a port in the biocompatible tubing interconnecting the serum lumen and the vacuum release lumen proximal to the distal tip; and
a body fluid lumen comprising a plurality of hollow fibers extending longitudinally within the serum lumen and configured to receive body fluid from the artery, vein or body fluid compartment.

2. The apparatus according to claim 1 further comprising:
a bidirectional volume displacement pump;
a connector configured for coupling the sampling catheter inserted in the artery, vein or body fluid compartment to the pump; and
a controller communicatively coupled to the pump that operates the pump at a predetermined flow rate to draw a body fluid volume into the body fluid lumen and that operates the pump at a predetermine flow rate to evacuate a body fluid volume from the body fluid lumen.

3. The apparatus according to claim 2 further comprising:
the controller configured to apply suction to the serum lumen wherein serum is drawn into the serum lumen through pores in the hollow fibers from the body fluid lumen and release suction through the vacuum release lumen.

4. The apparatus according to claim 1 further comprising:
the hollow fibers containing pores with diameter in a range of approximately 0.005-0.1 micrometers whereby body fluid components larger than the pore size are rejected.

5. The apparatus according to claim 1 further comprising:
a needle adapted for infusion therapy comprising a hypodermic tubing that encloses the serum lumen and the vacuum release lumen.

6. The apparatus according to claim 1 further comprising:
an analyte sensor configured for connecting to the sampling catheter and for mounting on a patient's body adjacent the sampling catheter comprising:
at least one infrared window;
a broadband infrared emitter that radiates through the at least one infrared window;
a sample chamber;
at least one narrowband filter in the emission path of the emitter; and
at least one detector that receives infrared signals passing through the at least one infrared window, the sample chamber, the at least one narrowband filter, the at least one detector coupled to transmit measurement signals to a display and control device.

7. The apparatus according to claim 1 further comprising:
an analyte sensor configured for connecting to the sampling catheter and accepting a sample of serum of less than 0.005 milliliter volume.

8. A serum sampling apparatus comprising:
a sampling catheter configured for insertion in an artery, vein or body fluid compartment comprising:
a biocompatible tubing extending longitudinally from a proximal end to a distal tip enclosing at least two lumens, the at least two lumens comprising:
a serum lumen circumferentially enclosed at least in part by a tubular membrane, the serum lumen extending to a solid wall at the distal tip;
a vacuum release lumen circumferentially enclosed within the biocompatible tubing and extending to a solid wall at the distal tip; and
a port in the biocompatible tubing interconnecting and enabling air flow between the serum lumen and the vacuum release lumen proximal to the distal tip.

9. The apparatus according to claim 8 further comprising:
the sampling catheter further comprising:
a porous support configured to support the membrane and prevent membrane collapse.

10. The apparatus according to claim 8 comprising:
a vacuum pump;
a connector that couples the serum lumen of the sampling catheter inserted in the artery, vein or body fluid compartment to the pump; and
a controller communicatively coupled to the pump that operates the pump to apply suction to the serum lumen and draw serum into the serum lumen through the membrane, and operates to release the vacuum through the vacuum release lumen.

11. The apparatus according to claim 8 further comprising:
an analyte sensor configured for coupling to and receiving a sample of filtered serum from the sampling catheter; and
a display and control device coupled to the analyte sensor that controls acquisition of serum, measurement of analyte concentration in the serum, and display of measurement results.

12. The apparatus according to claim 8 further comprising:
the tubular membrane having pores with diameter in a range of approximately 0.005-0.1 micrometers whereby body fluid components larger than the pore size are rejected.

13. An analyte concentration measurement apparatus comprising:
an implantable serum sampling tubing enclosing a vacuum release lumen and a serum lumen that are interconnected by a port, the serum lumen separated from the sampling tubing exterior by a membrane barrier, the sampling tubing configured for drawing a serum sample from a body fluid compartment by creating suction in the serum lumen;
an analyte sensor configured for coupling to and receiving a sample of filtered serum from the sampling tubing; and
a display and control device coupled to the analyte sensor that controls acquisition of serum, measurement of analyte concentration in the serum, and display of measurement results.

14. The apparatus according to claim 13 further comprising:
the display and control device comprising:
at least one pump with a volume displacement at least twice total interior volume of the serum lumen;
a control module that receives user commands from at least one input devices, and sends control signals to the analyte sensor and controls to the at least one pump;
a processor interfaced to the control module that processes analyte concentration measurement data for display;
a signal processing module that receives measurement signals from the analyte sensor and passes digitized analyte concentration measurement data to the processor; and
a display coupled to the processor that displays the processed absorption data.

15. The apparatus according to claim 13 further comprising:
the display and control device that operates in at least four modes including calibrate, infuse, sample, and measure modes:
the calibrate mode operation comprising infusing a known analyte concentration to the analyte sensor and calibrating the analyte sensor according to the known solution;
the infuse mode operation comprising infusing flush solution through the serum lumen into the body compartment; and
the sample mode operation comprising effusing a serum sample from the body compartment through the serum lumen to the analyte sensor; and
the measure mode operation comprising measuring the analyte concentration in the serum sample.

16. The apparatus according to claim 13 further comprising:
the display and control device configured to generate a display of the analyte concentration measurement in the serum sample within less than one minute of measurement initiation.

17. The apparatus according to claim 13 further comprising:
the display and control device further comprising a waste receptacle coupled to the serum sampling catheter configured for evacuating a sample of serum from the analyte sensor.

18. The apparatus according to claim 13 further comprising:
the sampling catheter configured for usage in fluid drainage, fluid injection, instrument access, and/or separation of serum from body fluid.

19. An implantable serum sampling apparatus comprising:
an infusion needle comprising at least one hypodermic tubing enclosing a plurality of internal lumens comprising a vacuum release lumen and a serum lumen that are interconnected by a port, and at least one body fluid lumen, the serum lumen separated from the at least one body fluid lumen by at least one hollow fiber configured to receive body fluid from the artery, vein, or body fluid compartment, the infusion needle configured for drawing a serum sample from the artery, vein, or body fluid compartment by creation suction in the serum lumen.

20. The apparatus according to claim 19 further comprising:
the hypodermic tubing extending longitudinally from a proximal end to a distal tip and comprising an outer tubing and an inner tubing positioned internal to the outer tubing, the hypodermic tubing distal tip configured for implanting in the artery, vein, or body fluid compartment;
the vacuum release lumen enclosed between the outer tubing and the inner tubing;
the at least one hollow fiber positioned internal to the inner tubing;
the serum lumen enclosed between the inner tubing and the at least one hollow fiber, the serum lumen and vacuum release lumen connected by the port at the distal tip enabling air flow therebetween;
the at least one body fluid lumen contained within the at least one hollow fiber and configured to flow body fluid from the artery, vein, or body fluid compartment, the at least one hollow fiber operative as a cross-flow separator that separates and filters body fluid from serum.

21. The apparatus according to claim 19 further comprising:
a volume displacement pump;
a connector configured for coupling the serum lumen of the infusion needle inserted in the artery, vein or body fluid compartment to the pump; and
a controller communicatively coupled to the pump that operates the pump at a predetermined flow rate to draw a body fluid volume into the at least one body fluid lumen, operates the pump at a predetermined flow rate to evacuate a body fluid volume from the at least one body fluid lumen, applies suction to the serum lumen wherein serum is drawn into the serum lumen through pores in the hollow fibers from the at least one body fluid lumen, and releases the suction through the vacuum release lumen.

22. The apparatus according to claim 19 further comprising:
the hollow fibers containing pores with diameter in a range of approximately 0.005-0.1 micrometers whereby body fluid components larger than the pore size are rejected.

23. A method for sampling and analyzing body fluid comprising:
providing a sampling catheter comprising a biocompatible tubing enclosing a vacuum release lumen and a serum lumen that are interconnected by a port, the serum lumen separated from a body compartment by a membrane barrier; and
drawing a serum sample from a body fluid compartment by creation of suction in the serum lumen.

24. The method according to claim 23 further comprising:
implanting the sampling catheter into a patient's vein, artery, or body fluid compartment;
connecting the sampling catheter to a sensor;
calibrating the sensor comprising infusing a known solution to the sensor and calibrating the sensor according to the known solution;
acquiring an analyte comprising effusing a body fluid sample from the vein or artery to the sensor;
measuring the analyte in the body fluid sample; and
flushing the sensor after each calibration and each measurement comprising infusing saline through the sensor and the sampling catheter into the vein or artery.

25. The method according to claim 23 further comprising:
connecting the sampling catheter to an analyte sensor, the sampling catheter comprising a tubing and a red blood cell-filtering barrier encasing the tubing that filters red blood cells from body fluid entering the tubing, the red blood cell-filtering barrier having pores of diameter in a range of approximately 0.005-0.1 micrometers whereby blood components larger than the pore size are rejected by the barrier;
mounting the analyte sensor on a patient's body adjacent the sampling catheter; and
drawing a sample from filtered body fluid of less than 1 milliliter volume for measurement.
